(19) United States
(12) Patent Application Publication
(10) Pub. No.: US 2008/0033068 A1
(43) Pub. Date: Feb. 7, 2008

(54) Production of Oxygenated Products

(75) Inventors: Petrus Wilhelmus Nicolaas Maria Van Leeuwen, Kockengen (NL); Edyta B. Walczuk, Lueneburg (DE); Neil Eugene Grimmer, Kempton Park (ZA); Paulas Clemens Jozef Kamer, St Andrews (GB)

Correspondence Address: LADAS & PARRY, 26 WEST 61ST STREET, NEW YORK, NY, 10023, US

(73) Assignee: SASOL TECHNOLOGY (PROPRIETARY) LIMITED, JOHANNESBURG (ZA)

(21) Appl. No.: 10/579,588

(22) PCT Filed: Nov. 17, 2004
(86) PCT No.: PCT/IB04/03758

§ 371 Date: Feb. 12, 2007

(30) Foreign Application Priority Data
Nov. 18, 2003 (ZA) 2003/8964

(51) Int. Cl. C07C 27/06

(52) U.S. Cl. 518/716

(57) ABSTRACT

A process for producing oxygenated products from a Fischer-Tropsch derived olefinic feedstock, includes reacting the feedstock, in a hydroformylation reaction stage, with carbon monoxide and hydrogen at an elevated reaction temperature and at a superatmospheric reaction pressure in the presence of a hydroformylation catalyst system. The catalyst system comprises a mixture, combination or complex of a transition metal, T, where T is selected from the transition metals of Group VIII of the Periodic Table of Elements; carbon monoxide, CO; hydrogen, H2; as a primary ligand, a monodentate phosphorus ligand; and as a secondary ligand, a bidentate phosphorus ligand which confers resistance on the catalyst system to poisoning arising from the presence of undesired components in the Fischer-Tropsch derived feedstock.

THIS INVENTION relates to the production of oxygenated products. It relates in particular to a process for producing oxygenated products from an olefinic feedstock.

The production of aldehydes and alcohols is conveniently accomplished by employing a transition metal hydroformylation catalyst, in the presence of carbon monoxide and hydrogen, to convert an olefinic substrate or feedstock. The use of a phosphorus containing compound as a component of the catalyst has been found to be highly beneficial in that higher product linearizes can be obtained under less severe operating conditions. A broad range of olefins can be hydroformylated using such transition metals modified with phosphorus containing ligands. However, it has been found that the olefinic feedstocks must be substantially pure, ie free of compounds such as dienes, ketones and alkynes. Such compounds are detrimental to the catalyst performance. These compounds may result in irreversible deactivation of the catalyst or have an incubatory effect from which the active hydroformylation catalyst may be recovered only if it is able to react with it. Invariably a dramatic drop, if not complete loss, of hydroformylation activity occurs as the catalyst is tied up with these incubated species due to their low reactivity. In particular, such compounds react, in preference to α-olefins, with the transition metal, to form species which react very slowly and therefore act as sinks removing catalyst from the hydroformylation process. Thus, a feedstock containing such components can have a catastrophic effect on a hydroformylation process where complete cessation of hydroformylation activity can result. These catalyst inhibitors must therefore be removed by means of purification procedures which are not only costly, but may result in the decrease of reactable α-olefins in the feedstock.

Fischer-Tropsch derived olefinic feedstocks are complex feedstocks and contain, in addition to α-olefins which are the desired olefins for hydroformylation to aldehydes and alcohols, also small amounts of other compounds such as other olefinic compounds, ie hydrocarbons having at least one double bond, being linear, branched or aromatic and not being α-olefins; dienes, conjugated and non-conjugated, with a terminal olefinic functionality or not; trienes; cyclic olefins; cyclic dienes; alkynes; ketones; aldehydes; esters; carboxylic acids and the like. Other feed components may include, where chemically possible, those consisting of combinations of these functionalities and/or combinations thereof with an α-olefin. As indicated hereinbefore, such compounds have hitherto been undesired in hydroformylation feedstocks, for the reasons given hereinbefore, and are hereinafter also referred to as ‘undesired components’.

By ‘Fischer-Tropsch derived’ in respect of the olefinic feedstock is meant that the feedstock has been obtained by the so-called Fischer-Tropsch process, ie obtained by reacting a synthesis gas comprising carbon monoxide and hydrogen in the presence of a suitable Fischer-Tropsch catalyst, normally a cobalt, iron, or cobalt/iron Fischer-Tropsch catalyst, at elevated temperature in a suitable reactor, which is normally a fixed, fluidized or slurry bed reactor, to obtain a range of products; these products must then be worked up to obtain a Fischer-Tropsch derived olefinic stream, typically a C₂ to C₂₀ olefinic stream, which is suitable for use as a feedstock to a hydroformylation process. This feedstock is characterized thereby that it has not been worked up sufficiently to remove all undesired components hereinbefore described, and is thus still a complex feedstock. For example, a typical Fischer-Tropsch derived olefinic stream that can be used as a complex feedstock to be converted by means of a hydroformylation reaction comprises 20-100 mass % paraffins and olefins, including α-olefins; 0-40 mass % aromatics; and 0-40 mass % oxygenates such as aldehydes, ketones, esters and carboxylic acids.

It is thus an aim of this invention to provide, in a hydroformylation process, a transition metal-ligand catalyst system by means of which such a complex Fischer-Tropsch derived olefinic feedstock can be hydroformylated directly. Thus, the catalyst system must either be more resistant to the negative influences of some of the undesired components in the feedstock or react more rapidly with them, than has hitherto been the case.

Thus, according to the invention, there is provided a process for producing oxygenated products from a Fischer-Tropsch derived olefinic feedstock, which process includes reacting the feedstock, in a hydroformylation reaction stage, with carbon monoxide and hydrogen at an elevated reaction temperature and at a superatmospheric reaction pressure in the presence of a hydroformylation catalyst system, which comprises a mixture, combination or complex of
(i) a transition metal, T, where T is selected from the transition metals of Group VII of the Periodic Table of Elements;
(ii) carbon monoxide, CO;
(iii) hydrogen, H₂;
(iv) as a primary ligand, a monodentate phosphorus ligand; and
(v) as a secondary ligand, a bidentate phosphorus ligand which confers resistance on the catalyst system to poisoning arising from the presence of undesired components in the Fischer-Tropsch derived feedstock.

The olefinic feedstock is thus a complex Fischer-Tropsch derived olefinic feedstock, as hereinbefore described, and typically contains, in addition to at least one α-olefin, a plurality of undesired components or compounds selected from another olefinic compound having at least one double bond, being linear, branched or aromatic and not being an α-olefin; a diene, conjugated or non-conjugated, conjugated, with a terminal olefinic functionality or not; a triene; a cyclic olefin; a cyclic diene; an alkyne; a ketone; an aldehyde; an ester; a carboxylic acid, and/or the like.

More particularly, T may be Rh, Co, Ir or Pd; however, rhodium (Rh) is preferred. Examples of rhodium sources that can be used are Rh(acac)(CO)₂ where ‘acac’ is acetylacetonate; Rh(acac)(CO)(TPP) where ‘acac’ is acetylacetonate; and ‘TPP’ is triphenylphosphine; [Rh(OAc)₂]₂ where ‘OAc’ is acetate; Rh₂O₃, Rh₄(CO)₁₂, Rh₆(CO)₁₆, Rh(CO)₂(dipivaloyl methanoate) or Rh(NO₃)₃. Preferably, the rhodium is initially in the form of Rh(acac)(CO)₂ or Rh(acac)(CO)(TPP).

The Applicant has found that it is beneficial to use, as the primary ligand in the hydroformylation catalyst system of a hydroformylation process according to the invention, a monodentate phosphorus ligand, in view of its ready availability; its relatively low cost; its ease of use, for example, the relatively low pressure at which the hydroformylation process can be conducted; and its robustness. However, the Applicant has also found that when such a monodentate ligand is used and when the feedstock is a Fischer-Tropsch derived olefinic feedstock which also contains undesired components as hereinbefore described, the undesired components react in preference to the α-olefin with the transition metal, to form chemical species which react very slowly and thus act as sinks for removing catalyst from the hydroformylation process, with potentially catastrophic consequences, as hereinbefore set out. In other words, when the catalyst contains only a monodentate phosphorus ligand, it is easily inhibited and poisoned by the plurality of undesired components in the complex Fischer-Tropsch derived feedstock.

However, the Applicant has unexpectedly found that the potentially harmful effects of such undesired components can be overcome or countered by adding to the hydroformylation catalyst system, as a secondary ligand, a bidentate phosphorus ligand. The bidentate phosphorus ligand thus confers resistance to the catalyst against the poisoning effect of the undesired components. The bidentate ligand, which is generally more expensive than the monodentate ligand, is used at a lower molar proportion than the monodentate ligand, relative to the transition metal.

The hydroformylation reaction stage may comprise a hydroformylation reactor. The process may then include initially preparing the catalyst system by dissolving component (i), together with the ligands, in a solvent, to produce a catalyst solution. This catalyst solution can then be introduced into the reactor, and upon heating thereof in the reactor in the presence of synthesis gas comprising CO and H₂, an active hydroformylation catalyst system is formed.

The concentration of rhodium in the catalyst solution in the hydroformylation reactor may be from 10 to 1000 ppm, more preferably from 50 and 500 ppm, and most preferably from 50 and 300 ppm.

The ligands are thus used in an excess molar concentration relative to the transition metal. The monodentate phosphorus ligand may be used in a molar excess, relative to the transition metal, of at least 20:1, typically from 20:1 to 2000:1, more preferably from 50:1 to 1000:1. It can even be used in a molar excess, relative to the transition metal, of from 90:1 to 1000:1. The bidentate phosphorus ligand will, in many circumstances, preferentially bind to the transition metal, displacing the monodentate ligand. It is employed at lower ligand to transition metal ratios such as at least 0.2:1, typically 0.2:1-100:1, and more preferably from 0.5:1-50:1, relative to the transition metal. The relative quantities of monodentate and bidentate phosphorus ligands used may be such that the molar proportion of bidentate ligand to monodentate ligand is not more than 0.2:1, and can be 0.1:1 or less. In some cases, the molar proportion of bidentate ligand to monodentate ligand can be 0.0555:1 or less, for example 0.03:1 or less, or even 0.018:1 or less. Of course, in each catalyst system an ideal monodentate:bidentate:transition metal ratio would have to be determined depending not only on the properties of the ligands employed, but also the product specification desired as well as the composition of the feedstock.

The monodentate phosphorus ligand is thus a chelating agent having a single group capable of attachment to the transition metal. In particular, it may be a monodentate phosphine or phosphite ligand. In one embodiment of the invention, the monodentate phosphorus ligand may be that of Formula (L1a), where ‘L1a’ is derived from Ligand 1a:

P(R^{a})(R^{a})(R^{a}) (L1a)

where all R^{a} are the same or are dissimilar, and are each a branched or straight chain alkyl or aryl radical. Preferably, however, each R^{a} is an aryl group, and all R^{a} are the same. Most preferably, each R^{a} may be phenyl so that the ligand of formula (L1a) is then triphenylphosphine (‘TPP’).

However, in another embodiment of the invention, the monodentate ligand may be that of Formula (L1b), where ‘L1b’ is derived from Ligand 1b:

P(OR^{a})(OR^{a})(OR^{a}) (L1b)

where R^{a} is as hereinbefore defined. Preferably, however, each R^{a} in Formula (L1b) is an aryl group, and all R^{a} are the same. Most preferably, each R^{a} may be a substituted phenyl ring. Thus, ligand of formula (L1b) may for example be tris(2,4-ditertiary butylphenyl) phosphite or tris(2-tertiary butylphenyl) phosphite.

The bidentate phosphorus ligand (which is hereinafter also generally referred to as L2) is thus a chelating agent having two groups capable of attachment to the transition metal. It may, in one embodiment of the invention, be in accordance with Formula (L2a):

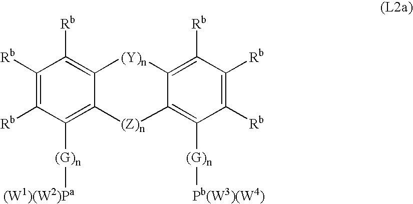

wherein
(i) all R^{b} are the same or are dissimilar, and are each H, alkyl, alkoxy, cycloalkyl, cycloalkoxy, heterocycloalkyl, aryl, heteroaryl, aryloxy, polyether, cyano, nitro, halogen, trifluoromethyl, —C(O)R^{c}, —(R^{d})C(O)R^{c}, —CHO, (R^{d})CHO, —COOR^{c}, —(R^{d})COOR^{c}, —COO⁻M⁺, —(R^{d})COO⁻M⁺, —SO₃R^{c}, —(R^{d})SO₃R^{c}, —SO₃⁻¹M⁺, —(R^{d})SO₃⁻M⁺, —SR^{c}, R^{d})SR^{c}, —SOR^{c}, —R^{d}(SOR^{c}), —NR^{c}, —(R^{d})NR^{c}, —N⁺(R^{c})(R^{c})(X⁻) or —(R^{d})N⁺(R^{c})(R^{c})(X⁻), wherein
   (a) R^{c} and R^{d} are the same or different, and are each H, or a branched or straight chain alkyl, alkoxy, cycloalkyl, polyether, cycloalkoxy, heterocycloalkyl, aryl, heteroaryl or aryloxy radical;
   (b) M⁺ is a cation; and
   (c) X⁻ is an anion;
(ii) Y and Z are independent bridges, are the same or different, and are each selected from the radicals —O—, —N(R^{c})—, —N⁺(R^{c})(R^{c})(X⁻)—, —N(C(O)R^{c})—, —C(R^{c})(R^{c})—, —C(C(R^{c})(R^{c}))—, —C(O)—, —S—, —Si(R^{c})(R^{c})—, —Si(OR^{c})(OR^{c})—, —P(R^{c})— or —P(OR^{c})—, where R^{c} and X⁻ are as hereinbefore defined;
(iii) n (in (Y)ₙ and (Z)ₙ) is, in each case, 0 or 1, with the proviso that n cannot be 0 for both Y and Z;
(iv) W¹, W², W³ and W⁴ are the same or different, and are each an alkyl (branched or straight chain), alkoxy, cycloalkyl, cycloalkoxy, heterocycloalkyl, aryl, heteroaryl, aryloxy or trifluoromethyl radical;
(v) a, b, in P^{a} and P^{b}, are used merely to identify the P atoms;
(vi) each G is an independent linker radical, are the same or different, and is selected from —O—, —N(R^{f})—, —N⁺(R^{f})(R^{f})(X⁻)—, —C(R^{f})(R^{f})—, —S—, —Si(R^{f})(R^{f})—, —C(F₂)— or —C(R^{f})(F)—, wherein
   (a) R^{f} is H, or a branched or straight chain alkyl, alkoxy, cycloalkyl, polyether, cycloalkoxy, heterocycloalkyl, aryl, heteroaryl or aryloxy radical, and with the proviso that when the radical contains more than one R^{f}, all R^{f} are the same or different;
   (b) X⁻ is as hereinbefore defined; and
(vii) n (in each (G)ₙ) is 0 or 1.

M⁺ may be an ion of an alkali or alkali earth metal, such as sodium, potassium or barium, or it may be ammonium or a quaternary ammonium ion.

X⁻ may be an organic acid, phosphate or sulphate group, for example —CO₂⁻, —PO₃²⁻ or —SO₃⁻.

When n=0, in (Y)n, then the independent Y bridge is naturally absent. L2 will then be in accordance with formula (L2b):

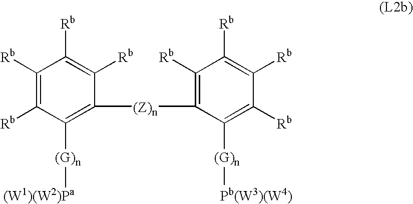

When n=0, in (Z)n, then the independent Z bridge is naturally absent. L2 will then be in accordance with formula (L2b):

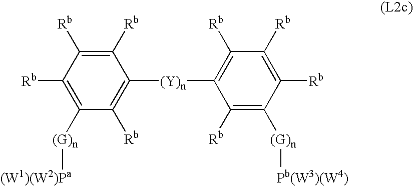

In L2 in accordance with formulae (L2b) and (L2c), R^{b}, W¹, W², W³, W⁴, Y, Z and G are thus as hereinbefore defined.

W¹, W², W³ and W⁴ may, in particular, each be an alkyl, aryl or aryloxy radical; however, aryl and aryloxy radicals are preferred. Most preferred are aryl or aryloxy radicals represented by Formula (1); however, the structure of Formula (1) does not represent a bridging unit connecting P^{a} to P^{b}—for P^{a}, W¹ and W² represent radicals connected through their respective G linkers, and for P^{b}, W³ and W⁴ represent radicals connected through their respective G linkers; however, for W¹ and W² on the one hand, and W³ and W⁴ on the other hand, G may be the same or different to that in L2 of Formula (L2a).

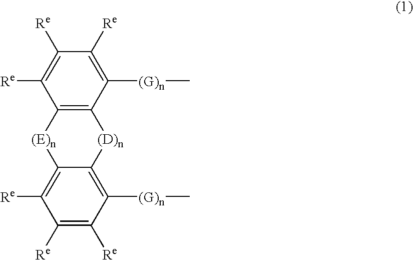

wherein
(i) all R^{e} are the same or different, and are each H, alkyl, alkoxy, cycloalkyl, cycloalkoxy, heterocycloalkyl, aryl, heteroaryl, aryloxy, polyether, cyano, nitro, halogen, trifluoromethyl, —C(O)R^{c}, —(R^{d})C(O)R^{c}, —CHO, (R^{d})CHO, —COOR^{c}, —(R^{d})COOR^{c}, —COO⁻M⁺, —(R^{d})COO⁻M⁺, —SO₃R^{c}, —(R^{d})SO₃R^{c}, —SO₃⁻M⁺, —(R^{d})SO₃⁻M⁺, —SR^{c}, —(R^{d})SR^{c}, —SOR^{c}, —R^{d}(SOR^{c}), —NR^{c}, —(R^{d})NR^{c}, —N⁺(R^{c})(R^{c})(X⁻) or —(R^{d})N⁺(R^{c})(R^{c})(X⁻), wherein R^{c}, R^{d}, X⁻ and M⁺ are as hereinbefore defined;
(ii) G and n (in (G)n) are as hereinbefore defined;
(iii) D and E are each an independent bridge, are the same or different, and are each selected from the radical, —O—, —N(R^{c})—, —N⁺ (R^{c})(R^{c})(X⁻), —N(C(O)R^{c}), —N(SiR₂^{c})—, —C(R^{c})(R^{c})—, —C(C(R^{c})(R^{c}))—; —C(O)—, —S—, —Si(R^{c})(R^{c})—, —Si(OR^{c})(OR^{c})—, P(R^{c}) or —P(OR^{c}), wherein R^{c} and X⁻ are as hereinbefore defined;
(iv) n (in each of (D)n and (E)n) is 0 or 1.

When D and/or E is present, W¹ and W² represent one diradical connected to the P atom. The same applies for W³ and W⁴.

When n=0, in (E)n, then the independent E bridge is naturally absent. The structure of formula (1) will then have the structure in accordance with formula (2):

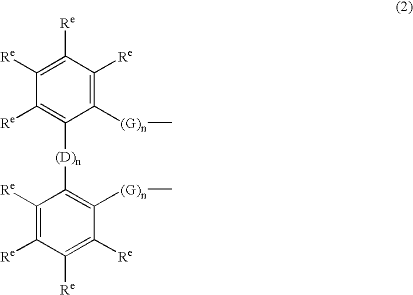

When n=0, in (D)n, then the independent D bridge is naturally absent. The structure of formula (1) will then have the structure in accordance with formula (3):

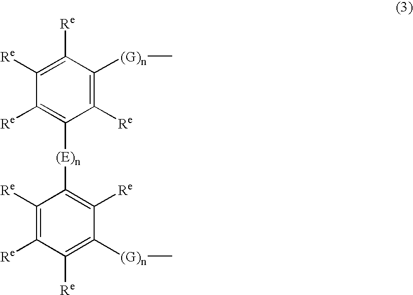

When n=0, in both (D)n and (E)n, then both the independent bridges D and E are naturally absent. The structure of formula (1) will then have the structure in accordance with formula (4) i.e. two separate, unbridged radicals:

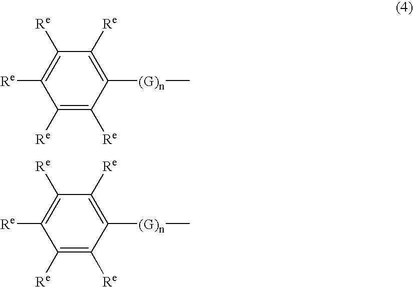

In formulae (2), (3) and (4), R^{e}, D, E and G are as hereinbefore defined.

When n=0, in respect of (G)n, then a direct connection exists between the P atom and the phenyl ring in the structures of formulae (L2a), (1), (2), (3) and (4). In such case, a phosphine substructure may be formed.

However, in, another embodiment of the invention, L2 may be that of formula (L2d):

(W¹)(W²)P^{a}—(G)ₙ—(A)—(G)ₙ—P^{b}(W³)(W⁴) (L2d)

where
(i) P, G, W¹, W², W³ and W⁴ are as hereinbefore defined;
(ii) A is a bridging unit and is selected from one of the following diradicals: —(CR^{b}₂)ₙ—, —CR^{b})ₙ—, —CR^{b}CR^{b})ₙ—, —[C(O)]ₙ—, —[C(O)C(R^{b})₂]ₙ—, —(NR^{b})ₙ—, —S—, —(SiR^{b}₂)ₙ—, —(SiOR^{b}₂)ₙ—, with
(a) any alkyl radical having n=1 to 5 and being cyclic, straight or branched or straight;
(b) R^{b} being as hereinbefore defined; or

(iii) A is a bridging unit and is ‘—Ar—’, which is an aryl or hereroaryl group of between 4 and 18 carbon atoms.

When, in formula (L2d), n=0 (in respect of (G)n), then a direct connection exists between a phosphorus atom and the bridging unit A in formula (L2d). In such case, a phosphine substructure may be formed.

In formulae (L2b), (L2c), (1), (2) and (3), the independent bridges X, Y, D and E may represent a direct linkage between the phenyl rings without an intervening group or atom.

Ideally, the bidentate ligand, L2, should have a wide bite angle, with those belonging to the xantphos family of ligands, and variations thereupon, being preferred examples. Examples of such preferred ligands L2 are given in formulae (L2e) to (L2n) below and in which Ph is C₆H₅ and ^{t}Bu is C(CH₃)₃:

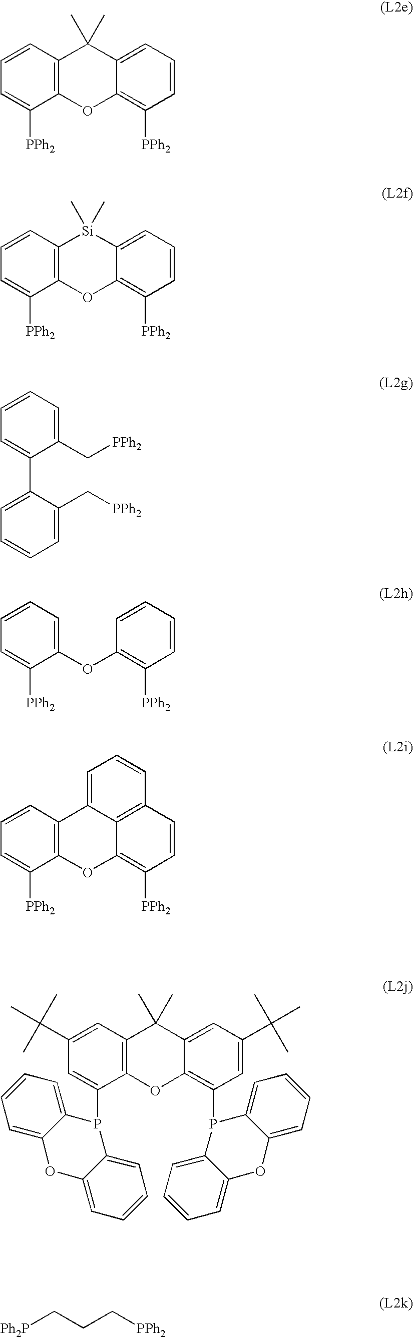

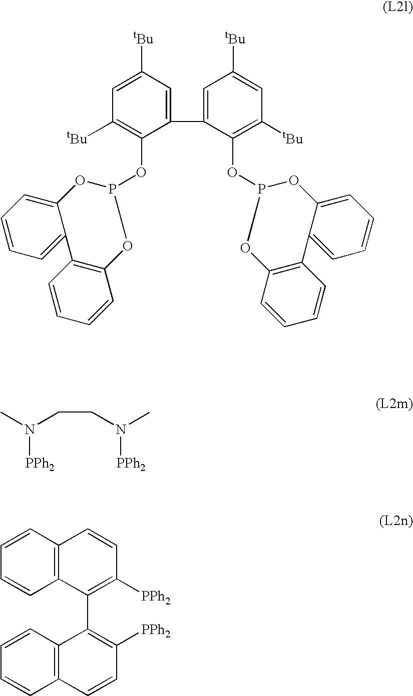

The reaction temperature in the hydroformylation reactor may be from 50° C. to 150° C., more preferably from 70° C. to 120° C.

The synthesis gas pressure under which the hydroformylation reaction is performed may be from 1 to 100 bar, but more preferably from 5 to 40 bar, and most preferably from 10 to 30 bar. The H₂:CO ratio may be from 1:10 to 100:1, but most preferably from 1:1 and 5:1.

The invention will now be described in more detail with reference to the following non-limiting examples:

In all the Examples, autoclave experiments were performed in Parr autoclaves. The catalyst precursors were dissolved in toluene in a Schlenk tube under an argon atmosphere. This solution was then transferred via cannula to the autoclave which had been purged of air with argon. The reactor was sealed, flushed twice with synthesis gas and then pressurised with synthesis gas. The reactor contents were then heated and upon reaching reaction temperature the substrate, ie the olefinic feedstock, was charged into the reactor, via a sample bomb, using synthesis gas overpressure. The progress of the reaction was monitored either by means of a mass-flow meter or the drop in pressure of a ballast vessel. Unless stated otherwise a 1:1 H₂:CO synthesis gas mixture was employed in the experiments. All ratios or proportions are given on a molar basis, unless otherwise specified.

### EXAMPLE 1

### Example 1a

Rh(acac)(CO)₂ (9.6×10⁻⁵ mol) and TPP (Rh:TPP=1:170) were dissolved in 50 ml toluene, which was then transferred to a 100 ml reactor. 1-Octene (10 ml) spiked with methyl vinyl ketone (100 mol eq. to Rh) was injected into the reactor once reaction temperature had been reached. The methyl vinyl ketone spiked 1-octene thus simulated a Fischer-Tropsch derived olefinic feedstock. The reaction was performed at 15 bar pressure and 100° C.

The time taken to reach 50% olefin conversion was 1 hr 45 min.

### Example 1b

The same experimental procedure as described Comparative Example 1a was followed with the difference that 4,5-bis(diphenylphosphino)-9,9-dimethylxanthene (hereinafter referred to as xantphos) was added as a secondary ligand (Rh:TPP:Xantphos=1:170:5).

The time taken to reach 50% olefin conversion was 1 hr.

### Example 1c

The same procedure as described for Example 1b was followed with the difference that the Rh:TPP:Xantphos ratio was changed to 1:170:3. The time taken to reach 50% olefin conversion was 1 hr.

### Example 1d

The same procedure as described for Example 1b was followed with the difference that the Rh:TPP:Xantphos ratio was changed to 1:170:1. The time taken to reach 50% olefin conversion was 1 hr 3 min.

### Example 1e

The same procedure as described for Example 1b was followed with the difference that the Rh:TPP:Xantphos ratio was changed to 1:90:5. The time taken to reach 50% olefin conversion was 35 min.

### EXAMPLE 2

### Example 2a

Rh(acac)(CO)₂ (9.6×10⁻⁵ mol) and TPP (Rh:TPP=1:170) were dissolved in 50 ml toluene, which was then transferred to a 100 ml reactor. 1-Octene (10 ml) spiked with isoprene (100 mol eq. to Rh) was injected into the reactor once reaction temperature had been reached. The isoprene spiked 1-octene thus simulated a Fischer-Tropsch derived olefinic feedstock. The reaction was performed at 15 bar pressure and 100° C.

The rate of hydroformylation, between 0-50% olefin conversion, was compared to a similar reaction where no isoprene was added and it was found that the diene had inhibited the reaction by 51%.

### Example 2b

The same experimental procedure as described Comparative Example 2a was followed with the difference that xantphos was added as a secondary ligand (Rh:TPP:Xantphos=1:170:5).

At 0-50% olefin conversion no catalyst inhibition was recorded when compared to a similar reaction where no diene had been added.

### Example 2c

The same experimental procedure as described in Example 2a was followed with the difference that (oxydi-2,1-phenylene)bis(diphenylphosphine) (hereinafter referred to as DPEphos) was added as a secondary ligand rather than xantphos (Rh:TPP:DPEphos=1:170:3).

At 0-50% olefin conversion 16% catalyst inhibition was recorded when compared to a similar reaction where no diene had been added.

### EXAMPLE 3

In a series of experiments the influence of a pure feed (dodecene-paraffin solution; 1:1) and a complex Fischer-Tropsch derived olefinic feed (C11/12 fraction) on different rhodium hydroformylation catalysts were evaluated and compared. The dodecene was diluted with an inert C9-11 paraffin to give a solution with a similar reactable olefin content to that of the Fischer-Tropsch derived feed. The Fischer-Tropsch derived olefinic feed had the following composition (on a mass basis): 53% paraffins and olefins, including α-olefins, internal linear olefins, branched internal and terminal olefins, dienes, trienes, cyclic olefins and cyclic dienes; 24% aromatics; and 23% oxygenates, including ketones, aldehydes, esters and carboxylic acids. Rh(acac)(CO)₂ (6×10⁻⁵ mol) and TPP (Rh:TPP=1:90) were dissolved in 30 ml toluene together with a bidentate ligand (Rh:bidentate=1:5), selected from Formulae I-VI, in which Ph is C₆H₅ and ^{t}Bu is C(CH3)₃, and the reactor prepared as described hereinbefore. The hydroformylation reaction was commenced by charging an olefin mixture consisting of hexene (10 ml) and either the dodecene-paraffin solution or Fischer-Tropsch feed (30 ml) into the reactor by means of synthesis gas overpressure on a sample vessel connected to the reactor. The reaction was carried out at 20 bar.

The productivity of the catalyst system under investigation was determined by sampling the reactor contents and determining the amount of hexene converted to aldehyde by GC-FID analysis of these samples. By comparing the difference in 1-hexene conversion after 0.5 hr, for the catalyst exposed to pure and Fischer-Tropsch derived feed, it is possible to obtain a measure by which the catalyst has been inhibited by undesired components in the latter feed. The results from these studies are collected in Table 1.

TABLE 1

| | Primary | Secondary | Difference in 1-hexene |
|---|---|---|---|
| Entry | Ligand | Ligand | conversion/% |
| 1 | TPP | none | 17 |
| 2 | TPP | I | <1 |
| 3 | TPP | II | 3 |
| 4 | TPP | III | <1 |
| 5 | TPP | IV | 9 |
| 6 | TPP | V | <1 |
| 7 | TPP | VI | <1 |

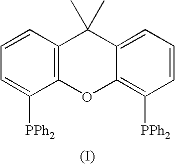

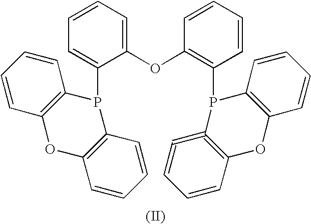

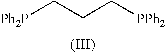

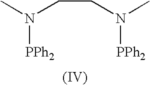

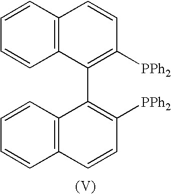

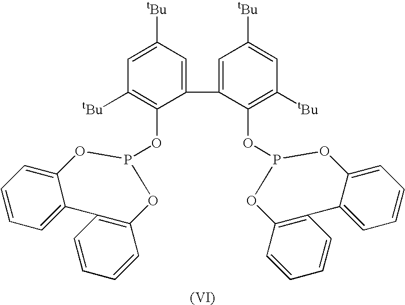

The Applicant has thus unexpectedly found that by using either a catalyst system comprising a Group VIII transition metal together with a monodentate phosphorus ligand/bidentate phosphorus ligand combination as hereinbefore described, in a hydroformylation process, an olefinic feedstock comprising at least one α-olefin and at least one undesired compound can be accommodated in the process. Thus, such an olefinic feedstock can then be treated in the hydroformylation process without unacceptable deactivation and/or loss of activity of the catalyst occurring.

1. A process for producing oxygenated products from a Fischer-Tropsch derived olefinic feedstock, which process includes reacting the feedstock, in a hydroformylation reaction stage, with carbon monoxide and hydrogen at an elevated reaction temperature and at a superatmospheric reaction pressure in the presence of a hydroformylation catalyst system, which comprises a mixture, combination or complex of
(i) Rh(acac)(CO)₂ where ‘acac’ is acetylacetonate, Rh(acac)(CO)(TPP) where ‘acac’ is acetylacetonate and ‘TPP’ is triphenylphosphine, [Rh(OAc)₂]₂ where ‘OAc’ is acetate, Rh₂O₃, Rh₄(CO)₁₂, Rh₆(CO)₁₆, Rh(CO)₂(dipivaloyl methanoate), or Rh(NO₃)₂;′
(ii) carbon monoxide, CO;
(iii) hydrogen, H₂;
(iv) as a primary ligand, a monodentate phosphorus ligand; and
(v) as a secondary ligand, a bidentate phosphorus ligand which confers resistance on the catalyst system to poisoning arising from the presence of undesired components in the Fischer-Tropsch derived feedstock.

2. A process according to claim 1, wherein the hydroformylation reaction stage comprises a hydroformylation reactor, with the process including initially preparing the catalyst system by dissolving component (i), together with the ligands, in a solvent, to produce a catalyst solution, and heating the catalyst solution in the reactor in the presence of synthesis gas comprising CO and H₂ to form an active hydroformylation catalyst system in which the rhodium concentration in the catalyst solution in the hydroformylation reactor is from 10 to 1000 ppm.

3. A process according to claim 1, wherein the monodentate phosphorus ligand is used in a molar excess, relative to the rhodium, of from 50:1 to 1000:1.

4. A process according to claim 1, wherein the bidentate phosphorus ligand is employed at a lower ligand to rhodium molar ratio than the monodentate phosphorus ligand, and wherein the bidentate phosphorus ligand to rhodium ratio is from 0.2:1 to 100:1.

5. A process according to claim 1, wherein the monodentate phosphorus ligand is
P(R^{a})(R^{a})(R^{a}) (L1a)
where all R^{a} are the same or are dissimilar, and are each a branched or straight chain alkyl or aryl radical.

6. A process according to claim 5, wherein in the ligand of formula (L1a), each R^{a} is an aryl group and all R^{a} are the same.

7. A process according to claim 6 wherein, in the ligand of formula (L1a), each R^{a} is phenyl so that ligand (L1a) is triphenylphosphine.

8. A process according to claim 1, wherein the monodentate phosphorus ligand is
P(OR^{a})(OR^{a})(OR^{a}) (L1b)
where all R^{a} are the same or are dissimilar, and are each a branched or straight chain alkyl or aryl radical.

9. A process according to claim 8, wherein in the ligand of formula (L1b), each R^{a} is an aryl group and all R^{a} are the same.

10. A process according to claim 9, wherein in the ligand of formula (L1b), each R^{a} is a substituted phenyl ring.

11. A process according to claim 10, wherein the ligand (L1b) is tris(2,4-ditertiary butylphenyl) phosphite or tris(2-tertiary butylphenyl) phosphite.

12. A process according to claim 1, wherein the bidentate phosphorus ligand is 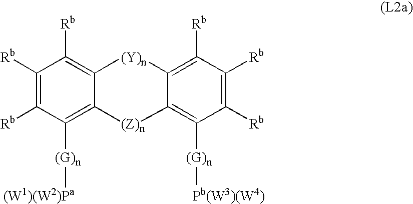
wherein
(i) all R^{b} are the same or are dissimilar, and are each H, alkyl, alkoxy, cycloalkyl, cycloalkoxy, heterocycloalkyl, aryl, heteroaryl, aryloxy, polyether, cyano, nitro, halogen, trifluoromethyl, —C(O)R^{c}, —(R^{d})C(O)R^{c}, —CHO, (R^{d})CHO, —COOR^{c}, —(R^{d})COOR^{c}, —COO⁻M⁺, —(R^{d})COO⁻M⁺, —SO₃R^{c}, —(R^{d})SO₃R^{c}, —SO₃⁻M⁺, —(R^{d})SO₃⁻M⁺, —SR^{c}, —(R^{d})SR^{c}, —SOR^{c}, —R^{d}(SOR^{c}), —NR^{c}, —(R^{d})NR^{c}, —N⁺(R^{c})(R^{c})(X⁻) or —(R^{d})N⁺(R^{c})(R^{c})(X⁻), wherein
(a) R^{c} and R^{d} are the same or different, and are each H, or a branched or straight chain alkyl, alkoxy, cycloalkyl, polyether, cycloalkoxy, heterocycloalkyl, aryl, heteroaryl or aryloxy radical;
(b) M⁺ is a cation; and
(c) X⁻ is an anion;
(ii) Y and Z are independent bridges, are the same or different, and are each selected from the radicals —O—, —N(R^{c})—, —N⁺(R^{c})(R^{c})(X⁻)—, —N(C(O)R^{c})—, —C(R^{c})(R^{c})—, C(C(R^{c})(R^{c}))—, —C(O)—, —S—, —Si(R^{c})(R^{c})—, —Si(OR^{c})(OR^{c})—, —P(R^{c})— or P(OR^{c})—, where R^{c} and X⁻ are as hereinbefore defined;
(iii) n (in (Y)ₙ and (Z)ₙ) is, in each case, 0 or 1, with the proviso that n cannot be 0 for both Y and Z;
(iv) W¹, W², W³ and W⁴ are the same or different, and are each an alkyl (branched or straight chain), alkoxy, cycloalkyl, cycloalkoxy, heterocycloalkyl, aryl, heteroaryl, aryloxy or trifluoromethyl radical;
(v) a, b, in P^{a} and P^{b}, are used merely to identify the P atoms;
(vi) each G is an independent linker radical, are the same or different, and is selected from —O—, —N(R^{f})—, —N⁺(R^{f})(R^{f})(X⁻)—, —C(R^{f})(R^{f})—, —S—, —Si(R^{f})(R^{f})—, —C(F₂)— or —C(R^{f})(F)—, wherein
(a) R^{f} is H, or a branched or straight chain alkyl, alkoxy, cycloalkyl, polyether, cycloalkoxy, heterocycloalkyl, aryl, heteroaryl or aryloxy radical, and with the proviso that when the radical contains more than one R^{f}, all R^{f} are the same or different;
(b) X⁻ is as defined above; and
(vii) n (in each (G)ₙ) is 0 or 1.

13. A process according to claim 1, wherein the bidentate phosphorus ligand is 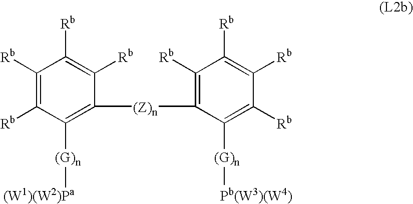
wherein
(i) all R^{b} are the same or are dissimilar, and are each H, alkyl, alkoxy, cycloalkyl, cycloalkoxy, heterocycloalkyl, aryl, heteroaryl, aryloxy, polyether, cyano, nitro, halogen, trifluoromethyl, —C(O)R^{c}, —(R^{d})C(O)R^{c}, —CHO, (R^{d})CHO, —COOR^{c}, —(R^{d})COOR^{c}, —COO⁻M⁺, —(R^{d})COO⁻M⁺, —SO₃R^{c}, —(R^{d})SO₃R^{c}, —SO₃⁻M⁺, —(R^{d})SO₃⁻M⁺, —SR^{c}, —(R^{d})SR^{c}, —SOR^{c}, —R^{d}(SOR^{c}), —NR^{c}, —(R^{d})NR^{c}, —N⁺(R^{c})(R^{c})(X⁻) or —(R^{d})N⁺(R^{c})(R^{c})(X⁻), wherein
(a) R^{c} and R^{d} are the same or different, and are each H, or a branched or straight chain alkyl, alkoxy, cycloalkyl, polyether, cycloalkoxy, heterocycloalkyl, aryl, heteroaryl or aryloxy radical;
(b) M⁺ is a cation; and
(c) X⁻ is an anion;
(ii) Z is an independent bridge, and is selected from the radicals —O—, —N(R^{c})—, —N⁺(R^{c})(R^{c})(X⁻)—, —N(C(O)R^{c})—, —C(R^{c})(R^{c})—, —C(C(R^{c})(R^{c}))—, —C(O)—, —S—, —Si(R^{c})(R^{c})—, —Si(OR^{c})(OR^{c})—, —P(R^{c})— or P(OR^{c})—, where R^{c} and X⁻ are as defined above;
(iii) n (in (Z)ₙ) is 1;
(iv) W¹, W², W³ and W⁴ are the same or different, and are each an alkyl (branched or straight chain), alkoxy, cycloalkyl, cycloalkoxy, heterocycloalkyl, aryl, heteroaryl, aryloxy or trifluoromethyl radical;
(v) a, b, in P^{a} and P^{b}, are used merely to identify the P atoms;
(vi) each G is an independent linker radical, are the same or different, and is selected from —O—, —N(R^{f})—, —N⁺(R^{f})(R^{f})(X⁻)—, —C(R)(R^{f})—, —S—, —Si(R^{f})(R^{f})—, —C(F²)— or —C(R^{f})(F)—, wherein
(c) R^{f} is H, or a branched or straight chain alkyl, alkoxy, cycloalkyl, polyether, cycloalkoxy, heterocycloalkyl, aryl, heteroaryl or aryloxy radical, and with the proviso that when the radical contains more than one Rf, all R^{f} are the same or different;
(d) X⁻ is as defined above; and
(vii) n (in each (G)ₙ) is 0 or 1.

14. A process according to claim 1, wherein the bidentate phosphorus ligand is 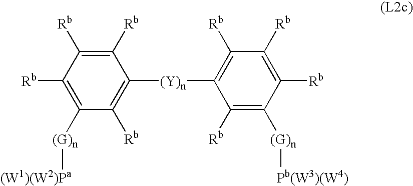
wherein
(i) all R^{b} are the same or are dissimilar, and are each H, alkyl, alkoxy, cycloalkyl, cycloalkoxy, heterocycloalkyl, aryl, heteroaryl, aryloxy, polyether, cyano, nitro, halogen, trifluoromethyl, —C(O)R^{c}, —(R^{d})C(O)R^{c}, —CHO, (R^{d})CHO, —COOR^{c}, —(R^{d})COOR^{c}, —COO⁻M⁺, —(R^{d})COO⁻M⁺, —SO₃R^{c}, —(R^{d})SO₃R^{c}, —SO₃⁻M⁺, —(R^{d})SO₃⁻M⁺, —SR^{c}, —(R^{d})SR^{c}, —SOR^{c}, —R^{d}(SOR^{c}), —NR^{c}, —(R^{d})NR^{c}, —N⁺(R^{c})(R^{c})(X⁻) or —(R^{d})N⁺(R^{c})(R^{c})(X⁻), wherein
(a) R^{c} and R^{d} are the same or different, and are each H, or a branched or straight chain alkyl, alkoxy, cycloalkyl, polyether, cycloalkoxy, heterocycloalkyl, aryl, heteroaryl or aryloxy radical;
(b) M⁺ is a cation; and
(c) X⁻ is an anion;
(ii) Y is an independent bridge, and is selected from the radicals —O—, —N(R^{c})—, —N⁺(R^{c})(R^{c})(X⁻)—, —N(C(O)R^{c})—, —C(R^{c})(R^{c})—, C(C(R^{c})(R^{c}))—, —C(O)—, —S—, —Si(R^{c})(R^{c})—, —Si(OR^{c})(OR^{c})—, —P(R^{c})— or P(OR^{c})—, where R^{c} and X⁻ are as hereinbefore defined;
(iii) n (in (Y)ₙ) is 1;
(iv) W¹, W², W³ and W⁴ are the same or different, and are each an alkyl (branched or straight chain), alkoxy, cycloalkyl, cycloalkoxy, heterocycloalkyl, aryl, heteroaryl, aryloxy or trifluoromethyl radical;
(v) a, b, in P^{a} and P^{b}, are used merely to identify the P atoms;
(vi) each G is an independent linker radical, are the same or different, and is selected from —O—, —N(R^{f})—, —N⁺(R^{f})(R^{f})(X⁻)—, —C(R^{f})(R^{f})—, —S—, —Si(R^{f})(R^{f})—, —C(F₂)— or —C(R^{f})(F)—, wherein
(e) R^{f} is H, or a branched or straight chain alkyl, alkoxy, cycloalkyl, polyether, cycloalkoxy, heterocycloalkyl, aryl, heteroaryl or aryloxy radical, and with the proviso that when the radical contains more than one R^{f}, all R^{f} are the same or different;
(f) X⁻ is as defined above; and
(vii) n (in each (G)ₙ) is 0 or 1.

15. A process according to claim 12, wherein in the ligand (L2a), M⁺ is an ion of an alkali or alkali earth metal, or is ammonium or a quaternary ammonium ion.

16. A process according to claim 12, wherein in the ligand (L2a), X⁻ is an organic acid, phosphate or sulphate group.

17. A process according to claim 12, wherein in the ligand (L2a), W¹, W², W³ and W⁴ are each an alkyl, aryl or aryloxy radical.

18. A process according to claim 17, wherein in the ligand (L2a), W¹, W², W³ and W⁴ are each an aryl or aryloxy radical in accordance with formula (1), with the proviso that the structure of formula (1) does not represent a bridging unit connecting P^{a} to P^{b}—for P^{a}, W¹ and W² represent radicals connected through their respective G linkers, and for P^{b}, W³ and W⁴ represent radicals connected through their respective G linkers 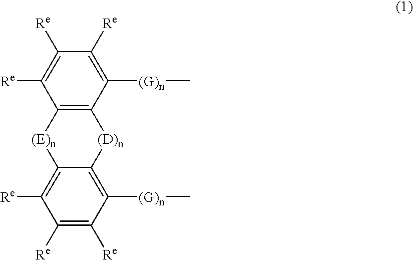
wherein
(i) all R^{e} are the same or are different, and are each H, alkyl, alkoxy, cycloalkyl, cycloalkoxy, heterocycloalkyl, aryl, heteroaryl, aryloxy, polyether, cyano, nitro, halogen, trifluoromethyl, —C(O)R^{c}, —(R^{d})C(O)R^{c}, —CHO, (R^{d})CHO, —COOR^{c}, —(R^{d})COOR^{c}, —COO⁻M⁺, —(R^{d})COO⁻M⁺, —SO₃R^{c}, —(R^{d})SO₃R^{c}, —SO₃⁻M⁺, —(R^{d})SO₃⁻M⁺, —SR^{c}, —(R^{d})SR^{c}, —SOR^{c}, —R^{d}(SOR^{c}), —NR^{c}, —(R^{d})NR^{c}, —N⁺(R^{c})(R^{c})(X⁻) or —(R^{d})N⁺(R^{c})(R^{c})(X⁻), wherein
(a) R^{c} and R^{d} are the same or different, and are each H, or a branched or straight chain alkyl, alkoxy, cycloalkyl, polyether, cycloalkoxy, heterocycloalkyl, aryl, heteroaryl or aryloxy radical;
(b) M⁺ is a cation; and
(c) X⁻ is an anion;
(ii) each G is an independent linker radical, are the same or different, and is selected from —O—, —N(R^{f})—, —N⁺(R^{f})(R^{f})(X⁻)—, —C(R^{f})(R^{f})—, —S—, —Si(R^{f})(R^{f})—, —C(F²)— or —C(R^{f})(F)—, wherein
(d) R^{f} is H, or a branched or straight chain alkyl, alkoxy, cycloalkyl, polyether, cycloalkoxy, heterocycloalkyl, aryl, heteroaryl or aryloxy radical, and with the proviso that when the radical contains more than one R^{f}, all R^{f} are the same or different;
(e) X⁻ is as defined above; and
(iii) n (in each (G)ₙ) is 0 or 1;
(iv) D and E are each an independent bridge, are the same or different, and are each selected from the radical, —O—, —N(R^{c})—, —N⁺(R^{c})(R^{c})(X⁻), —N(C(O)R^{c})—, —N(SiR₂^{C})—, —C(R^{c})(R^{c})—, —C(C(R^{c})(R^{c}))—; —C(O)—, —S—, —Si(R^{c})(R^{c})—, —Si(OR^{c})(OR^{c})—, —P(R^{c})— or —P(OR^{c})—, wherein R^{c} is H, or a branched or straight chain alkyl, alkoxy, cycloalkyl, polyether, cycloalkoxy, heterocycloalkyl, aryl, heteroaryl or aryloxy radical, and X− is as defined above;
(v) n (in each of (D)n and (E)n) is 0 or 1.

19. A process according to claim 18 wherein, in formula (1), n=0, in (E)ₙ, so that the independent E bridge is absent; formula (1) will then have the structure of formula (2)

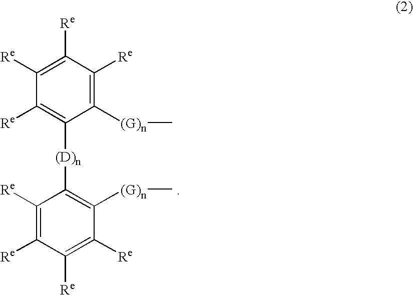

20. A process according to claim 18 wherein, in formula (1), n=0, in (D)n, so that the independent D bridging is absent; formula (1) will then have the structure of formula (3)

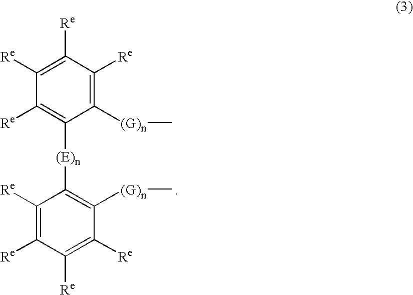

21. A process according to claim 18, wherein, in formula (1), n=0, in both (D)n and (E)n, so that both the independent bridges D and E are absent; formula (1) will then have the structure of formula (4)

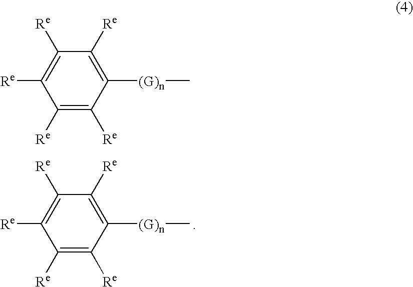

22. A process according to claim 1, wherein the bidentate phosphorus ligand is
(W¹)(W²)P^{a}—(G)ₙ—(A)—(G)ₙ—P^{b}(W³)(W⁴) L2d)
wherein
(i) each G is an independent linker radical, are the same or different, and is selected from —O—, —N(R^{f})—, —N⁺(R^{f})(R^{f})(X⁻)—, —C(R^{f})(R^{f})—, —S—, —Si(R^{f})(R^{f})—, —C(F²)— or —C(R^{f})(F)—, wherein
(a) R^{f} is H, or a branched or straight chain alkyl, alkoxy, cycloalkyl, polyether, cycloalkoxy, heterocycloalkyl, aryl, heteroaryl or aryloxy radical, and with the proviso that when the radical contains more than one R^{f}, all R^{f} are the same or different;
(b) X⁻ is an anion; and
(ii) n (in each (G)ₙ) is 0 or 1;
(iii) a, b, in P^{a} and P^{b}, are used merely to identify the P atoms;
(iv) W¹, W², W³ and W⁴ are the same or different, and are each an alkyl (branched or straight chain), alkoxy, cycloalkyl, cycloalkoxy, heterocycloalkyl, aryl, heteroaryl, aryloxy or trifluoromethyl radical; and
(v) A is a bridging unit and is selected from one of the following diradicals: —(CR^{b}₂)ₙ—, —(CR^{b})ₙ—, —(CR^{b}CR^{b})ₙ—, —[C(O)]ₙ—, —[C(O)C(R^{b})₂]ₙ—, —(NR^{b})ₙ—, —S—, —(SiR^{b}₂)ₙ—, —(SiOR^{b}₂)ₙ—, with
(c) any alkyl radical having n=1 to 5 and being cyclic, straight or branched or straight;
(d) R^{b} being H, alkyl, alkoxy, cycloalkyl, cycloalkoxy, heterocycloalkyl, aryl, heteroaryl, aryloxy, polyether, cyano, nitro, halogen, trifluoromethyl, —C(O)R^{c}, —(R^{d})C(O)R^{c}, —CHO, (R^{d})CHO, —COOR^{c}, —(R^{d})COOR^{c}, —COO⁻M⁺, —(R^{d})COO⁻M⁺, —SO₃R^{c}, —(R^{d})SO₃R^{c}, —SO₃⁻M⁺, —(R^{d})SO₃⁻M⁺, —SR^{c}, —(R^{d})SR^{c}, —SOR^{c}, —R^{d}(SOR^{c}), —NR^{c}, —(R^{d})NR^{c}, —N⁺(R^{c})(R^{c})(X⁻) or —(R^{d})N⁺(R^{c})(R^{c})(X⁻), wherein
(e) R^{c} and R^{d} are the same or different, and are each H, or a branched or straight chain alkyl, alkoxy, cycloalkyl, polyether, cycloalkoxy, heterocycloalkyl, aryl, heteroaryl or aryloxy radical;
(f) M⁺ is a cation; or
(vi) A is a bridging unit and is ‘—Ar—’, which is an aryl or hereroaryl group of between 4 and 18 carbon atoms.

23. A process according to claim 1, wherein the reaction temperature is from 50° C. to 150° C.; the synthesis gas pressure under which the hydroformylation reaction is performed is from 1 to 100 bar; and the H₂:CO ratio is from 1:10 to 100:1.
